(19) United States
(12) Patent Application Publication
(10) Pub. No.: US 2007/0048370 A1
(43) Pub. Date: Mar. 1, 2007

(54) Pharmaceutical formulation for salts of monobasic acids with clopidogrel

(76) Inventors: Karl-Heinz Doser, Buchholz i.d. Nordheide (DE); Klaus Glanzer, Hamburg (DE); Uwe Loffler, Tornesch (DE)

Correspondence Address: SMITH PATENT CONSULTING CONSULTING, LLC, 3309 DUKE STREET, ALEXANDRIA, VA, 22314, US

(21) Appl. No.: 11/392,824

(22) Filed: Mar. 30, 2006

(30) Foreign Application Priority Data
Sep. 1, 2005 (DE) DE 202005013839.8

(51) Int. Cl. A61K 9/20; A61K 31/4743

(52) U.S. Cl. 424/464; 514/301

(57) ABSTRACT

A pharmaceutical formulation in the form of a tablet which contains, as active substance, a salt of a monobasic acid with clopidogrel is disclosed herein.

The present invention relates to a pharmaceutical formulation in the form of a tablet which contains, as active substance, a salt of a monobasic acid with clopidogrel.

Clopidogrel (5-methyl-a-(4,5,6,7-tetrahydro[2,3-c]thienopyridyl)(2-chlorophenyl)acetate) is disclosed as an active substance in EP-A-0 99 802. Clopidogrel acts as a platelet aggregation inhibitor and can therefore be used, for example, for preventing thromboembolic events, such as, for example, stroke or myocardial infarction.

The active substance clopidogrel is used as the bisulfate in the commercial pharmaceutical formulations. Owing to the acidic proton in the bisulfate anion, clopidogrel bisulfate is strongly acidic.

Plavix 75 mg film-coated tablets registered in Europe contain clopidogrel bisulfate as active substance, and lactose, mannitol, Macrogol 6000, microcrystalline cellulose, hydrogenated caster oil, hydroxypropylcellulose having a low degree of substitution, hypromellose, triacetin, Carnauba wax, titanium dioxide and iron (III) oxide as excipients.

JP 3397385 describes clopidogrel-containing pharmaceutical formulations which comprise sucrose fatty acids for improving the disintegration time. The formulations also contain polyethylene glycol 6000 (PEG 6000, Macrogol 6000).

EP 1 178 809 describes the composition and production of oral combination preparations which contain clopidogrel bisulfate and aspirin.

EP 1 310 245 discloses clopidogrel bisulfate tablets in which magnesium stearate is replaced by zinc stearate, stearic acid or sodium stearyl fumarate.

WO 2005/070464 discloses clopidogrel bisulfate tablets which contain hydrogenated vegetable oils as lubricants.

To date, the prior art has been concerned with only a few non-bisulfate salts of clopidogrel. For example, WO 2004/072084 and WO 2004/072085 disclose general formulations comprising various sulfonic acid salts of clopidogrel, but without going into details.

WO 2004/074215 describes oral formulations, inter alia comprising clopidogrel mesylate and clopidogrel hydroiodide. All tablet formulations comprising clopidogrel mesylate contain PEG 6000, magnesium stearate and/or talc.

It has been found that pharmaceutical excipients as usually used for the active substance clopidogrel bisulfate are only poorly suitable for the formulation of a salt of a monobasic acid with clopidogrel, since they lead to decomposition of the active substance and hence to poor stability of the formulation. An object of the present invention is therefore to provide a pharmaceutical formulation for a salt of a monobasic acid with clopidogrel, which formulation does not have said disadvantages. In particular, the pharmaceutical formulation should be stable even during prolonged storage and should prevent decomposition of the active substance. In addition, the formulation should permit rapid and virtually quantitative release of the active substance.

It has now surprisingly been found that numerous excipients which are successfully used in practice for the formulation of clopidogrel bisulfate are incompatible with clopidogrel salts of a monabasic acid and promote decomposition of the active substance. It has been found that, for example, Macrogol 6000, which is used as an excipient in the Plavix tablets containing clopidogrel bisulfate, is incompatible with clopidogrel besylate and leads to decomposition of the active substance. Magnesium stearate, one of the most commonly used and most effective lubricants, croscarmellose sodium (AcDiSol), one of the most widely used disintegrants, and sodium lauryl sulfate, one of the most commonly used wetting agents, also proved to be incompatible with clopidogrel besylate and led to decomposition of the active substance. In comparison, with nonionic and/or nonbasic tabletting excipients, it was possible to obtain stable pharmaceutical formulations even with salts of a monobasic acid with clopidogrel.

The present invention therefore relates to pharmaceutical formulations in the form of a tablet, containing, as active substance, a salt of a monobasic acid with clopidogrel or the solvates or hydrates thereof, with the proviso that the salt is not clopidogrel hydroiodide, wherein the tablet contains no ionic and/or basic tabletting excipient and/or polyethylene glycol 6000.

Without wishing to be tied to a theory, it is assumed that ionic and basic compounds adversely effect the stability of a salt of a monobasic acid with clopidogrel, since these compounds release the clopidogrel base from the salts of monobasic acids, and the clopidogrel base is very susceptible to decomposition. In contrast, the bisulfate salt has a second, very acidic proton which is capable of buffering basic additives. Presumably, it is for this reason that additives such as zinc stearate, magnesium stearate, croacarmellose sodium, sodium lauryl sulfate and magnesium stearyl fumarate, the anionic moiety of which reacts as a Brönstedt base, can be used in pharmaceutical formulations comprising clopidogrel bisulfate. In contrast salts of clopidogrel with monobasic acid have no second, acidic group. Presumably, these salts are therefore very sensitive to basic additives.

It is also known that polyethylene glycols can undergo transesterification reactions (H. P. Fiedler (ed), Lexikon der Hilfsstoffe [Lexicon of excipients], Editio Cantor Veriag Aulendorf, 4^{th} edition 1998). It is assumed that Macrogol 6000 is therefore incompatible with clopidogrel besylate—clopidogrel is an ester—and leads to decomposition of the active substance. Surprisingly, this incompatibility with clopidogrel bisulfate does not occur. The formulation according to the invention therefore preferably contains no polyethylene glycol.

In particular, clopidogrel hydrochloride, hydrobromide, mesylate, besylate, benzoate, salicylate, lactate and gluconate are suitable as a salt of a monobasic acid with clopidogrel. Clopidogrel besylate is particularly preferred. Clopidogrel hydroiodide is excluded here owing to its poor pharmacological activity.

Basic tableting excipients are understood in the present application as meaning those excipients which have a pH of ≧7 in water. These include talc having a specific pH range of 7-9 (Pharm. Eur. 2002: 0438 Talc, 4^{th} edition).

Tableting excipients which may be used are in particular disintegrants, wetting agents, lubricants, binders, fillers and flow regulators. Suitable disintegrants are, for example, starches, modified starches, PVPs and modified PVPs. Suitable wetting agents are, for example, partial fatty acid esters of sorbitan (SPANs), partial fatty acid esters of polyhydroxyethylenesorbitan (TWEENs), polyhydroxyethers and polyhydroxyesters (Chremophor, etc.). Suitable lubricants include, for example, stearic acid and fumaric acid. Suitable binders and fillers are, for example, lactoses, celluloses, hydroxypropylcellulose, hydroxypropylmethylcelluose, polyols (e.g. Mannitol) and starches. Flow regulators which may be used are, for example, silica and titanium dioxide.

Optionally clopidogrel salts with monobasic acids may be further stabilized be the addition of acidic excipients. Not limited examples of acidic excipients are citric acid and benzene sulfonic acid.

The tablets according to the invention preferably comprise a tablet core obtained by direct compression. The tablet may be coated with a suitable film. Appropriate film coats are known to the person skilled in the art.

The pharmaceutical formulation according to the invention should ensure rapid and virtually quantitative release of the active substance. Preferably, more than 50%, preferably more than 80% and particularly preferably more than 95% of the active substance are released in vitro at pH 1.0, measured according to Pharm. Eur. (paddle, 75 rpm, 900 ml, 37° C.), in 15 minutes.

The present invention is explained in more detail by the following examples, without limiting it thereto.

### EXAMPLE 1

The stability of clopidogrel besylate in the presence of various tabletting excipients was investigated. For this purpose, clopidogrel besylate was thoroughly triturated with the excipient in the ratio 1:1 and stored for two weeks at 40° C. and 75% relative humidity. The content of clopidogrel besylate in the sample was then determined. The results are summarized in the following table, which indicates the clopidogrel content at the end of the storage time in comparison to the beginning of the storage, in percent.

TABLE

| | Clopidogrel besylate |
|---|---|
| Excipient | Residual content (%) |
| — | 99.76 |
| Macrogol 6000 | 90.07 |
| AcDiSol | 94.00 |
| Mg-stearate | 51.99 |
| Lactose | 99.86 |
| Mannitol | 99.85 |
| Stearic acid | 99.50 |
| Silica | 99.44 |
| Microcrystalline cellulose | 99.24 |

It is evident that ionic and basic tabletting excipients very considerably increase the stability of clopidogrel besylate, whereas nonionic and nonbasic tabletting excipients do not decompose clopidogrel besylate.

### EXAMPLE 2

The following two formulations are suitable for the production of tablet cores by direct compression:

### FORMULATION 1

| | | |
|---|---|---|
| Clopidogrel besylate | 111.86 | mg |
| Lactose monohydrate | 132.72 | mg |
| Cornstarch | 23.42 | mg |
| Colloidal SiO₂ | 2.50 | mg |
| Stearic acid | 4.5 | mg |

### FORMULATION 2

| | | |
|---|---|---|
| Clopidogrel besylate | 111.86 | mg |
| Mannitol | 109.3 | mg |
| Microcrystalline cellulose | 46.84 | mg |
| Colloidal SiO₂ | 2.50 | mg |
| Stearic acid | 4.5 | mg |

Hardness: approximately 60100 N, depending on the disintegration times

Disintegration times: not more than 5 min.

In vitro release (pH 1.0): >80% within 15 min.

The tablet can subsequently be coated with a suitable film.

1. A pharmaceutical formulation in the form of a tablet, containing, as active substance, a salt of a monobasic acid with clopidogrel or the solvates or hydrates thereof, with the proviso that the salt is not clopidogrel hydroiodide, wherein the tablet contains no ionic and/or basic tabletting excipient, and no polyethylene glycol 6000.

2. The pharmaceutical formulation of claim 1, wherein the clopidogrel salt of a monobasic acids is selected from the group consisting of clopidogrel hydrochloride, clopidogrel hydrobromide, clopidogrel mesylate, clopidogrel besylate, clopidogrel benzoate, clopidogrel salicylate, clopidogrel lactate, and clopidogrel gluconate.

3. The pharmaceutical formulation of claim 1, wherein the formulation includes a nonionic and/or nonbasic tabletting excipient selected from the group consisting of disintegrants, wetting agents, lubricants, binders, fillers and flow regulators.

4. The pharmaceutical formulation of claim 3, wherein the disintegrant is selected from the group consisting of starch, modified starch, PVP, modified PVP, and combinations thereof.

5. The pharmaceutical formulation of claim 3, wherein the wetting agent is selected from the group consisting of; a partial fatty acid ester of sorbitan (SPAN), a partial fatty acid ester of polyhydroxyethylenesorbitan (TWEEN), a polyhydroxyether, a polyhydroxyester (Chremophor), and combinations thereof.

6. The pharmaceutical formulation of claim 3, wherein the lubricant is selected from the group consisting of stearic acid, fumaric acid and combinations thereof.

7. The pharmaceutical formulation of claim 3, wherein the binder and/or filler is selected from the group consisting of lactose, cellulose, hydroxypropylcellulose, hydroxylpropylmethylcellulose, polyol, starch, and combinations thereof.

8. The pharmaceutical formulation of claim 3, wherein the flow regulators is selected from the group consisting of silica, titanium dioxide, and combinations thereof.

9. The pharmaceutical formulation of claim 1, wherein the formulation contains a tablet core obtained by direct compression and is optionally provided with a film coating.

10. The pharmaceutical formulation of claim 1, which releases more than 50% of the active substance in vitro at pH 1.0, measured according to Pharm. Eur. (paddle, 75 rpm, 900 ml, 37° C.), in 15 minutes.

11. The pharmaceutical formulation of claim 7, wherein the polyol comprises mannitol.

12. The pharmaceutical formulation of claim 1, which releases more than 80%, of the active substance in vitro at pH 1.0, measured according to Pharm. Eur. (paddle, 75 rpm, 900 ml, 37° C.), in 15 minutes.
